(19) United States
(12) Patent Application Publication
(10) Pub. No.: US 2008/0267955 A1
(43) Pub. Date: Oct. 30, 2008

(54) FRIZZLED 9 AS TUMOR MARKER

(75) Inventors: Hermann Schluesener, Weilheim (DE); Hans-Jorg Buhring, Tuebingen (DE); Sigrid Hojak, Berlin (DE); Ingrid Albert, Tuebingen (DE)

Correspondence Address: KLARQUIST SPARKMAN, LLP, 121 SW SALMON STREET, SUITE 1600, PORTLAND, OR, 97204, US

(73) Assignee: Eberhard-Karls-Universitaet Tuebingen Universitaetsklinikum, Tuebingen (DE)

(21) Appl. No.: 12/019,608

(22) Filed: Jan. 24, 2008

Related U.S. Application Data
(63) Continuation of application No. PCT/EP2006/007284, filed on Jul. 24, 2006.

(30) Foreign Application Priority Data
Jul. 25, 2005 (DE) 10 2005 035 568.4

(51) Int. Cl. A61K 39/395; G01N 33/574; C12Q 1/68; A61P 31/00

(52) U.S. Cl. 424/133.1; 424/138.1; 424/178.1; 435/7.23; 435/6

(57) ABSTRACT

The present invention relates to a method for treating and a method for identifying a tumor of a human or animal comprising administering composition comprising an agent which binds to Frizzled 9.

### CROSS-REFERENCES TO RELATED APPLICATIONS

This application is a continuation of international patent application PCT/EP/2006/007284 filed on Jul. 24, 2006, and designating the U.S., which international patent application has been published in German language as WO 2007/012449 A1, and claims priority of German patent application DE 10 2005 035 568.4 filed on Jul. 25, 2005. The entire contents of these priority applications are incorporated herein by reference.

### BACKGROUND OF THE INVENTION

The present invention relates to the use of an agent which binds to Frizzled 9 for producing a therapeutic or diagnostic composition for treating or identifying a tumor, to a process for producing a therapeutic or diagnostic composition for treating or identifying a tumor, to methods for identifying tumors, and to a method for treating a human or animal creature having a tumor.

### RELATED PRIOR ART

Detection of tissue-typical expression of a so-called biomarker or marker protein makes it possible for a histologist to identify particular tissues or organs of an organism. In addition, an abnormal pattern of expression of a biomarker frequently allows conclusions to be drawn about a pathological change in the affected tissue or organ or else about a disorder of the whole organism. Thus, for example, it is possible to conclude from overexpression of a proliferation-activating factor, e.g. certain cyclin-dependent kinases, that there is a neoplastic transformation of the cells affected thereby or, for instance, that there is a corresponding predisposition thereto. The same applies on underexpression or complete absence of the synthesis of tumor suppressor proteins such as, for example, the p53 protein.

Thus, biomarkers play a crucial role in particular in the area of the diagnosis and therapy of neoplastic diseases. It is often possible by qualitative or quantitative analysis of such biomarkers, which are also referred to as tumor markers in this connection, for the tumor to be identified and classified in relation to its origin or its existence.

Comprehensive knowledge about such biomarkers or tumor markers and their pattern of expression makes it possible not only to develop diagnostic methods but also to develop cell-targeted diagnostic or therapeutic agents.

However, the association of defined biomarkers with particular tissues also makes it possible to understand better the regulation of physiological processes. Thus, for example, expression of a key protein of signal transduction in proliferating endothelium indicates that the latter is involved in regulating angiogenesis.

Against this background, an object on which the present invention is based is to provide a biomarker or tumor marker by means of which in particular neurotumors/CNS tumors, prostate carcinomas and hemangiomas can be identified and, where appropriate, therapeutically treated. It is intended in particular to provide an agent which binds to such a biomarker and is suitable as active ingredient of a therapeutic or diagnostic composition.

This object is achieved by using an agent which binds to Frizzled 9 (FZD9) for producing a therapeutic or diagnostic composition for treating and/or identifying a tumor, where the tumor is selected from the group consisting of: neurotumor (CNS tumor), prostate carcinoma and breast carcinoma.

The suitability of Frizzled 9 for the identification and/or therapeutic treatment of these tumors was so surprising because the Frizzled proteins have generally been described in a different context in the prior art.

The proteins of the Frizzled (FZD) family belong to a group of receptors which have seven transmembrane segments. The FZD proteins are activated by so-called Wnt factors which form a family of signal molecules which undergo extracellular secretion. The N-terminal extracellular cystein-rich domain of FZD has in this connection been identified as Wnt-binding domain.

Wnt/FZD proteins play an important part in embryonic development of metazoa. They regulate a wide variety of developmental processes such as the specification, proliferation, migration and polarity of the cell. Combined with the low-density lipoprotein-like receptors 5 and 6, FZD proteins mediate at least three Wnt/FZD signaling pathways. The protein β-catenin is involved in one of these signaling pathways. This so-called β-catenin signaling pathway induces regulation of the transcription of various target genes, including c-Myc, cyclin D1, matrix metallo-proteinase 7 (MMP-7), immunoglobulin transcription factor 2 (ITF-2). The signaling pathway which regulates planar cell polarity is induced by Wnt binding via GTPase and activation of the c-jun amino-terminal kinase (JNK), leading to control of morphogenetic cell movement. The Ca²⁺ signaling pathway activates phospholipase Cβ and increases the intracellular Ca²⁺ level, resulting in activation of protein kinase C, of the Ca²⁺-calmodulin-dependent protein kinase II and of calcineurin, thus influencing the fate of the cell and the adhesion process. An overview of the Wnt/FZD signaling pathways is to be found in Huang H C, Klein P S, “The Frizzled family: receptors for multiple signal transduction pathways”, Genome Biol. 2004; 5:234-241 and in FIG. 1.

The human FZD 9 gene, originally referred to as FZD 3, is located in the genetically defined 1.4 Mb region on chromosome 7q11.23. The human FZD 9 protein consists of 591 amino acids.

Recently published experiments in which mice with an FZD 9 knockout mutation were produced indicate that FZD 9 plays a part in the blood-forming system; cf. Ranheim E A et al., “Frizzled 9 knock-out mice have abnormal B-cell development”, Blood. 2005; 105:2487-2494.

Wang et al. describe in “A novel human homologue of the Drosophila frizzled wnt receptor gene binds wingless protein and is in the Williams syndrome deletion at 7q11.23”, Hum. Mol. Genet. 1997; 6:465-472, by contrast, that FZD 9 is expressed in a large number of very diverse human tissues such as, for example, in the brain, but also in skeletal muscle, in the kidneys, the eyes and the testes.

In addition, Zhao C and Pleasure J, “Frizzled-9 promoter drives expression of transgenes in the medial wall of the cortex and its chief derivative the hippocampus”, Genesis 2004; 40:32-39, describe expression of FZD 9 extending widely over the brain, for example in the medial wall of the cortex, the telencephalon, the hippocampus and further regions of the mouse brain.

The unpublished German patent application DE 10 204 059 620 presents data showing that FZD 9 is expressed by a wide variety of different cell types such as, for example, leukemia cells, retinoblastoma cells or embryonal carcinoma cells.

It has therefore been assumed to date that FZD 9 is a ubiquitous protein which is wholly unsuitable as biomarker and in particular as tumor marker for the identification and/or therapeutic treatment of neurotumors/CNS tumors, prostate carcinomas and breast carcinomas.

Neurotumors/CNS tumors are divided according to the invention into (1.) primary brain-intrinsic brain tumors, (2.) spinally growing tumors and into (3.) tumors of peripheral nerves. The primary brain-intrinsic brain tumors in turn include the neuroepithelial tumors such as, for example, gliomas including astrocytomas, the embryonal tumors including medulloblastomas, the tumors of cerebral nerves including schwannomas and the tumors of the meningides including meningiomas. The spinally growing tumors may be differentiated into intramedullary and extramedullary tumors, with the extramedullary tumors possibly also including those which at the same time represent primary brain tumors such as the meningiomas or schwannomas. The extramedullary tumors also include hemangioblastoma, a benign tumor of the brain consisting of numerous smaller vessels (capillaries) and a soft tissue (stroma) located between them. Hemangioblastoma is associated in 25% of cases with the Hippel-Lindau syndrome (VHL) and may also occur multiply in the cerebellum and in the retina of the eye (called retinoblastoma there). Tumors of peripheral nerves may likewise include those which had already been mentioned in connection with the two classes described above, such as, for example, schwannomas. However, tumors of peripheral nerves also include neurofibromatoses.

Prostate carcinoma means according to the invention a tumorous dysplasia of the prostate. Breast carcinoma is used as synonym for breast cancer.

The inventors have realized for the first time that FZD 9 is a biomarker or tumor marker by means of which the identification and/or therapeutic treatment of neurotumors/CSN tumors, prostate carcinomas and breast carcinoma is possible.

An agent which binds to FZD 9 means according to the invention any means which is able to interact selectively or specifically with FZD 9. This interaction can preferably take place directly, although an indirect interaction is possible in turn with those factors which interact with FZD 9, such as, for example, the Wnt protein etc. (see above and FIG. 1). An agent which binds to FZD 9 therefore includes in the context of the invention binding proteins such as polyclonal or monoclonal antibodies, and antibody fragments which exhibit the binding properties of the antibodies. Such antibody fragments include so-called Fab fragments and so-called scFv fragments. A Fab fragment comprises the variable domain of the heavy and light chain and the adjoining constant domains C_{H}1 and C₁, connected by a disulfide bridge which naturally holds these two chains together. An scFv fragment consists of an Fv fragment, i.e. the smallest antibody domain which is (jointly) responsible for the affinity of the whole antibody for the antigen, and of a stabilized linker polypeptide. The specificities and selectivities of such Fab and scFv fragments are identical to those of the whole antibody. However, antibody fragments can be produced substantially more easily and thus more cost-effectively, for example in microbial expression systems.

A suitable polyclonal antibody against human FZD 9, which is employed in immunostaining, preferably in a dilution of 1:500, is marketed for example by Acris Antibodies GmbH, Hiddenhausen, Germany (catalog No. SP4153P). However, a suitable agent binding to FZD 9 is also according to the invention a nucleic acid molecule which is able to bind directly or indirectly and selectively or specifically to FZD 9 or to the factors mentioned, such as, for example, an aptamer.

It is particularly preferred in the use according to the invention to use as an agent which binds to FZD 9 a monoclonal antibody which is produced by the hybridoma cell W3C4E11 which was deposited on Jul. 15, 2004, in accordance with the Budapest Treaty at the DSMZ under the number DSM ACC2668.

This measure has the particular advantage that an agent which binds highly specifically to FZD 9 is used, in which case the identification or therapeutic treatment of the tumors mentioned then takes place by established immunological or molecular techniques. The inventors have realized for the first time that the monoclonal α-PZD 9 antibody which is known in principle from the unpublished German patent application DE 10 2004 050 620 is suitable as such an agent, with which the tumors under discussion can be identified and, where appropriate, treated. This suitability was unexpected in particular because the patent application presents data showing that this antibody binds to a wide variety of cell types.

The inventors have further found that the monoclonal antibody of the IgM isotype which is produced by the deposited W3C4E11 hybridoma cells binds not only to native tissue, for example of the human brain, but unexpectedly also to frozen, i.e. so-called cryotissue. Moreover, this monoclonal antibody is surprisingly not only suitable for identifying the tumors mentioned, but can also exert a direct effect on the tumors, for example on astocytomas, for example inhibit growth thereof. The deposited monoclonal antibody therefore also exhibits therapeutic potential.

It is preferred in this connection for the neurotumor/CNS tumor to be selected from the group consisting of glioblastomas including astrocytomas, medulloblastomas including primitive neuroectodermal tumors (PNET), schwannomas, hemangioblastomas, meningiomas and neurofibromatomas.

This measure has the advantage that an agent is provided for the identification or therapeutic treatment of particularly important tumors. Thus, for example, PNET is a tumor of embryonic origin which usually arises in the cerebellum as medulloblastoma and is one of the commonest malignant tumors occurring in children.

In another embodiment of the invention, it is preferred in the use according to the invention for the antibody to be a humanized antibody or a humanized antibody fragment.

Humanized antibodies, which are also referred to as recombinant antibodies or CDR-grafted antibodies, are antibodies in which the sequences for the hypervariable regions (CDRs) in the human immunoglobulin genes are replaced by the CDRs of immunoglobulin genes of other organisms, for example of the mouse. The antigenic specificity of an antibody, preferably of a mouse monoclonal antibody, is transferred by this humanization to a human antibody, thus generating—in the recipient organism—a complete tolerance to these molecules and avoiding a so-called HAMA response (human anti-mouse antibody response), through which a pure mouse antibody would be neutralized in humans because of the immune response, even after multiple administration.

It is preferred in a further embodiment of the present invention for the agent or the antibody or the antibody fragment to be coupled to a detectable marker and/or a therapeutic active substance.

A marker means according to the invention any compound by means of which it is possible to locate and identify the substructures of the human brain in vitro, in vivo or in situ. These include color indicators such as dyes having fluorescent, phosphorescent or chemiluminescent properties, AMPPD, CSPD, radioactive indicators such as ³²P, ³⁵S, ¹²⁵I, ¹³¹I, ¹⁴C, ³H, nonradioactive indicators such as biotin or digoxigenin, alkali phosphatase, horseradish peroxidase etc. Antibodies labeled in this way are then detected by means of imaging methods known in the prior art, such as autoradiography, blotting, hybridization or microscopic techniques.

The use according to the invention is developed through this measure in an advantageous manner such that a treatment or identification of the tumor is possible even more reliably and simply.

A suitable therapeutic active substance is in principle any active substance which induces a specific biological reaction in an organism or in a biological cell. Examples of particularly suitable therapeutic active substances are pharmaceuticals or toxins which inhibit the growth of the tumors, such as radiotherapeutic agents, chemotherapeutic agents or activators of the complement system or of apoptosis. An antibody coupled in this way can advantageously transport the active substance in a targeted manner into a tumor such as an astrocytoma, or into its blood-supplying system, and there induce destruction of the tumor or of the pathological blood vessels. It is also possible for psychopharmaceuticals to be transported by such an antibody via the hippocampus into the limbic system and there mediate a targeted effect of psychopharmaceuticals. Side effects of therapeutic active substances which are intended to act selectively in the human brain are reduced in this way.

Against this background, a further aspect of the present invention relates to a process for producing a therapeutic and/or diagnostic composition for the treatment or identification of a tumor with the steps: (1) provision of an agent which binds to FZD 9, (2) formulation of the agent in a diagnostically or pharmaceutically acceptable carrier with, where appropriate, further additives, where the tumor is selected from the group consisting of: neurotumor/CNS tumor, prostate carcinoma and breast carcinoma.

In relation to the agent which binds to FZD 9, all the features described above for the use of the latter according to the invention apply to the aforementioned process. The statements made above about the use according to the invention likewise apply to the tumors.

Diagnostically and pharmaceutically acceptable carriers with, where appropriate, further additives are generally known in the prior art and are described for example in the treatise by Kibbe A., “Handbook of Pharmaceutical Excipients”, Third Edition, American Pharmaceutical Assoc. and Pharmaceutical Press 2000. Additives include according to the invention any compound or composition which are advantageous for a diagnostic or therapeutic use of the composition, including salts, binders, but also further active substances.

A further aspect of the present invention relates to a method for identifying tumors in a creature which includes the following steps: (1) provision of a biological sample derived from the creature to be investigated; (2) establishment of the level of expression of FZD 9 in the biological sample; (3) comparison of the level of expression of FZD 9 established in step (2) with the level of expression in a biological sample from a healthy creature, and (4) identification of a tumor if it is established in step (3) that the level of expression of FZD 9 in the biological sample of the creature to be investigated is higher than the level of expression of FZD 9 in the biological sample of the healthy creature.

Identification means according to the invention the recognition or detection and, where appropriate, the classification of the tumors. For this purpose, the level of expression of FZD 9 in a biological sample derived from the possibly tumorous region of the creature is compared with the level of expression of FZD 9 in a reference sample derived from the corresponding region of a healthy creature. The extent to which FZD 9 is synthesized in the biological sample is established by means of methods known in the prior art, such as immunological, histological, imaging or microscopic methods.

As the inventors have surprisingly been able to establish, the level of expression of FZD 9 in tissues of a neurotumor/CNS tumor, prostate carcinoma or breast carcinoma is distinctly raised by comparison with the level of expression of FZD 9 in corresponding non-tumorous tissues of a healthy creature, such as, for example, in healthy brain/nerve tissue, healthy prostate tissue, healthy breast tissue etc.

Consequently, cells or tissues suitable according to the invention as biological sample are those which are to be investigated for the presence of a tumor. If it is intended to investigate for the presence of a neurotumor/CNS tumor, a biological sample which includes neurons, glia cells, meninges, and generally neural tissue or brain tissue is provided. If it is intended to investigate for the presence of a prostate carcinoma, a biological sample which includes prostate tissue or cells thereof is provided. If it is intended to investigate for the presence of breast carcinoma, a biological sample which includes breast tissue or cells thereof is provided. The reference sample used in each case is a sample derived from corresponding anatomical regions of a healthy creature.

It is possible to establish directly, by means of statistical methods known to the skilled worker, whether the increase in the level of expression in the biological sample of the creature to be investigated is significantly increased. Thus, a significant increase can be assumed when the level of expression of FZD 9 in the biological sample of the creature to be investigated is increased by only 5%, preferably 10%, more preferably by 50% by comparison with the level of expression of FZD 9 in the biological sample of the healthy creature.

The level of expression can be established both at the level of the mRNA coding for FZD 9 and at the level of the FZD 9 protein. In this connection, the level of expression at the mRNA level is established by means of methods known in the prior art, such as, for example, the Northern blot technique. At the protein level, the level of expression is preferably established by means of immunological techniques, for example by using the agent which binds to FZD 9 as described above in connection with the use according to the invention, for example a polyclonal or monoclonal antibody or a corresponding antibody fragment, which binds to FZD 9.

A further aspect of the present invention relates to methods for identifying tumors in a creature, which includes the following steps: (1) administration of a composition which includes an agent which binds to FZD 9 and which includes a detectable marker to the creature, and (2) demonstration of the accumulation of the agent in the creature by means of suitable methods, where the tumor is selected from the group consisting of neurotumors/CNS tumors, prostate carcinoma and breast carcinoma. The abovementioned detectable markers are suitable as detectable marker, this preferably being achieved by a radioactive marker, and the method in step (2) being 3D preferably radiography.

Three-dimensional demonstration of the tumor is possible by this imaging method, thus advantageously assisting the surgeon in the removal thereof.

A further aspect of the present invention relates to a method for treating a human or animal creature having a tumor, with the steps: (1) administration of a composition to the creature which comprises an agent which binds to FZD 9, and (2) where appropriate multiple repetition of step (1), where the tumor is selected from the group consisting of: neurotumor/CNS tumor, prostate carcinoma and breast carcinoma.

The inventors have further been able to establish that FZD 9 can be used for the identification and/or therapeutic treatment of substructures of the human brain, where the substructures are preferably proliferating cells, tumor cells, or astrocytoma cells. Angiogenesis, more preferably tumor angiogenesis, is recognized and/or therapeutically treated preferably via the identification and/or therapeutic treatment of the substructures. The identification and/or therapeutic treatment of the substructures preferably takes place by means of an antibody, preferably a monoclonal antibody, most preferably by means of one produced by the hybridoma cell W3C4E11 deposited in accordance with the Budapest treaty at the DSMZ under the number DSM ACC2668, or by means of a corresponding antibody fragment. The antibody may preferably be a humanized antibody. The antibody or the antibody fragment is preferably coupled to a marker or/and a therapeutic active substance.

The inventors have further developed a method for the identification and/or isolation of substructures of the human brain in a biological sample, which includes the following steps: (1) provision of the biological sample; (2) contacting the biological sample with an agent which binds selectively or/and specifically to FZD 9; (3) establishing whether a selective/specific binding of the agent to the substructure has taken place and (4) correlation of a positive establishment in step (3) with the identification of substructures of the human brain or/and (5) isolation of the substructures of the human brain in the event of a positive establishment in step (3). The substructures of the human brain are preferably proliferating cells, tumor cells, most preferably astrocytoma cells.

The inventors have further developed a method for identifying angiogenesis, preferably tumor angiogenesis, in a biological sample, which includes the following steps: (1) provision of the biological sample, preferably including tumor cells, (2) contacting the biological sample with an agent which selectivity or/and specifically binds to FZD 9, (3) establishing whether a selective/specific binding of the agent to the biological sample has taken place, and (4) correlation of a positive establishment in step (3) with the identification of angiogenesis in the biological sample. The statements made above about the agent which binds to FZD 9 in connection with the use according to the invention apply correspondingly to the agent.

The inventors have further realized that an agent directed against FZD 9, preferably an antibody, is suitable for producing a therapeutic or diagnostic composition for the treatment or diagnosis of a tumor, preferably a brain tumor, most preferably an astrocytoma.

The inventors have further surprisingly established that there is enhanced expression of FZD 9 in the pes hippocampi and the hippocampal neurons of the healthy, in particular human brain, and that the deposited monoclonal antibody is suitable for the identification and/or therapeutic treatment of hippocampal tissue via FZD 9. This measure therefore provides a use and a method which make simple and reliable identification, treatment and, where appropriate, isolation of human hippocampal tissue possible. This measure also has the advantage that the inventors for the first time provide information about the pattern of expression of FZD 9 in particular in the adult human brain, and thus disclose important information about possible signaling mechanisms in the hippocampus. The inventors therefore provide an important tool for identifying this region of the cerebral cortex not only for clinicians but also for cell biologists and fundamental researchers.

It will be appreciated that the features mentioned above and yet to be explained below can be used not only in the combination indicated in each case, but also in other combinations or alone, without departing from the scope of the present invention.

### BRIEF DESCRIPTION OF THE DRAWINGS

The present invention is now explained in more detail by means of exemplary embodiments which are purely illustrative in nature and which in no way restrict the scope of the present invention. Reference is made in this connection to the enclosed figures, which show:

FIG. 1 Wnt/FZD signaling pathway

FIG. 2 production of the antibody which is specifically directed against human FZD 9 and is derived from the W3C4E1 hybridoma cells. A: diagram which shows schematically the plasmid pIRES which includes the human FZD 9 gene and at the N terminus a Flag tag. B: FACS analysis of the specificity of the antibodies derived from the W3C4E11 cells. The monoclonal antibody derived from the W3C4E11 cells (right-hand part of the figure) and its nonspecific IgM isotype (left-hand part of the figure) were incubated with HEK-293 cells which were transfected with the pIRES plasmid depicted in (A). After washing, the cellular fluorescence intensity was analyzed by flow cytometry.

FIG. 3 immunohistochemistry for FZD 9 in normal human brains (A, B) and in human astrocytomas of WHO grade II (C), grade III (D) and grade IV (E-H). In normal brains, FZD 9 was located mainly in the neurons of the hippocampus (A+B). FZD 9 immunostaining was unambiguously detectable in the microvessels and the tumor cells of the astrocytomas with WHO grade II (C), grade III (D) and grade IV (E). FZD 9 was expressed in the endothelial cells in the microvessels (F). In the glioblastomas it was possible to stain the multilayer microvascular proliferations (‘glomeroid tufts’) (G) and necroses with pseudopalisades (‘pseudopalisading necrosis’) (H), which are histopathological characteristics of glioblastomas, strongly for FZD 9. Original magnifications: ×40 (A); ×400 (B−E, G); ×1000 (F); ×200 (H).

FIG. 4 statistical analysis (Pearson's correlation coefficient analysis) of the correlation between the immunoreactivity of FZD 9 with the astrocytoma classification, microvessel density and the astrocytoma Ki67 labeling index. The density of FZD 9⁺ microvessels correlated positively with the WHO astrocytoma classification (A) and the astrocytoma MVD (B) in human astrocytomas. The total FZD 9 immunostaining intensity correlated highly with the WHO astrocytoma classification (C) and the K167 labeling index (D) in human astrocytomas.

FIG. 5 changes in FZD 9 mRNA expression after cobalt chloride treatment in HCEC cells. The cells were incubated with cobalt chloride (100 μmol) for the stated time. FZD 9 expression was quantified by using real-time PCR. FZD 9 expression was raised 15 hours after incubation with cobalt chloride and distinctly raised 24 hours after the incubation.

FIG. 6 intensity of the immunohistochemical staining for FZD 9 in 23 samples of medulloblastomas compared with 10 samples of neuropathologically normal human brains.

FIG. 7 correlation of the intensity of the FZD 9 stain with the MIB-1 (Ki67) labeling index.

FIG. 8 combined real-time reverse transcription PCR analysis on four tissue samples which reflect FZD 9 mRNA expression in medulloblastomas compared with normal brain samples. The values are indicated in the form of the expression relative to 18S rRNA, which was used as internal reference standard.

FIG. 9 immunohistochemical expression of FZD 9. Cerebellar expression is confined to the endothelium of a few vessels (a, 100× magnification). In the telencephalon, FZD 9 is located mainly in the hippocampal neurons (d, 200× magnification). FZD 9 expression in the tumor vessels is indicated by arrows (c, 400× magnification). The strong expression (intensity=3) in a medulloblastoma (d, 100× magnification) and in supratentorial PNETs (e, 400× magnification) is depicted by way of example. Weak (value=1) staining (f, 100× magnification) and moderate (value=2) staining (g, 400× magnification) in medulloblastomas. A survey of the same tumor as shown in part g of the figure illustrates an unequal distribution pattern of FZD 9 expression (h, 100× magnification).

FIG. 10 intensity of the immunohistochemical staining for FZD 9 in a schwannoma sample.

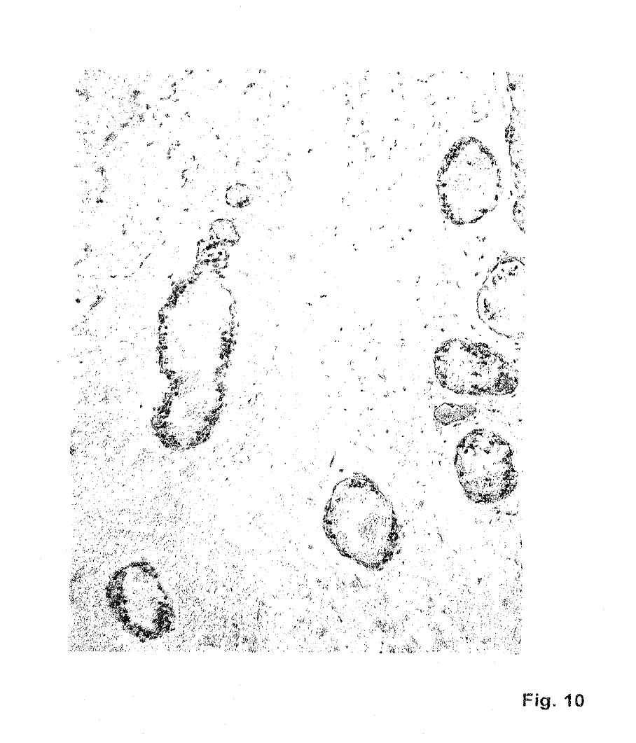

FIG. 11 intensity of the immunohistochemical staining for FZD 9 in a meningioma sample.

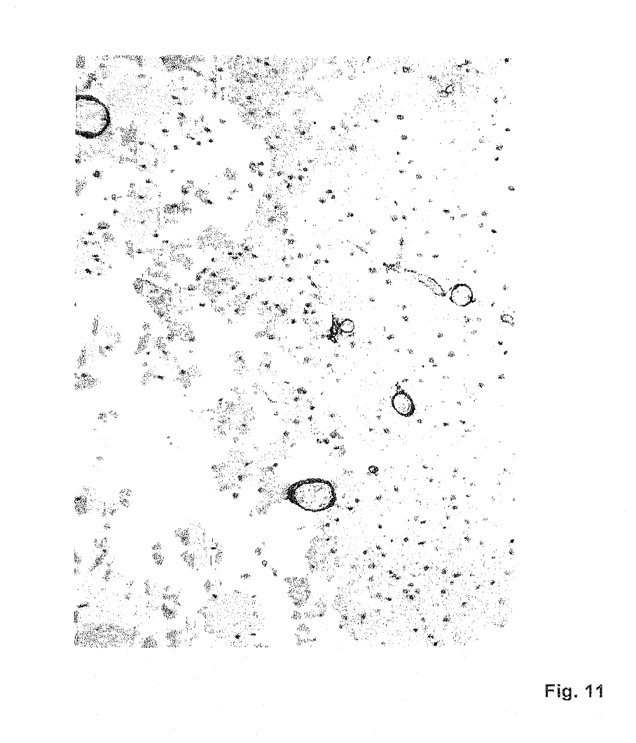

FIG. 12 intensity of the immunohistochemical staining for FZD 9 in a neurofibromatoma sample.

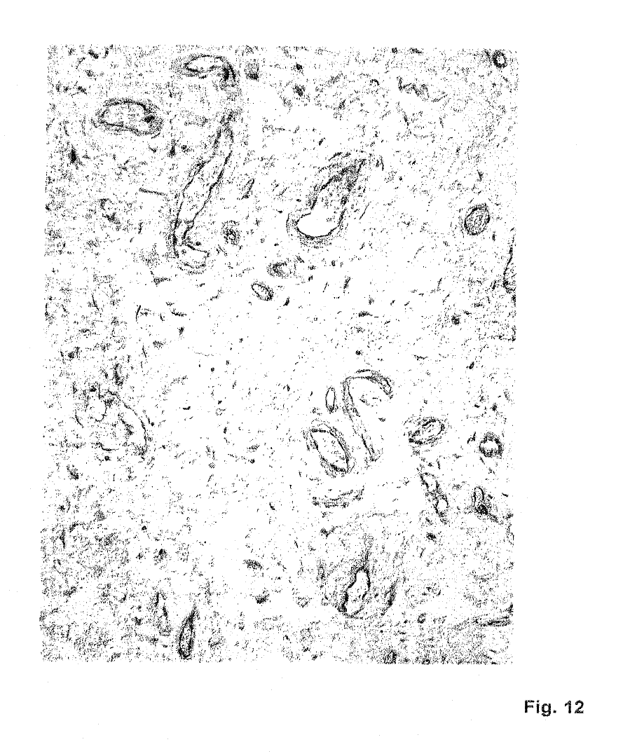

FIG. 13 intensity of the immunohistochemical staining for FZD 9 in a prostate carcinoma sample.

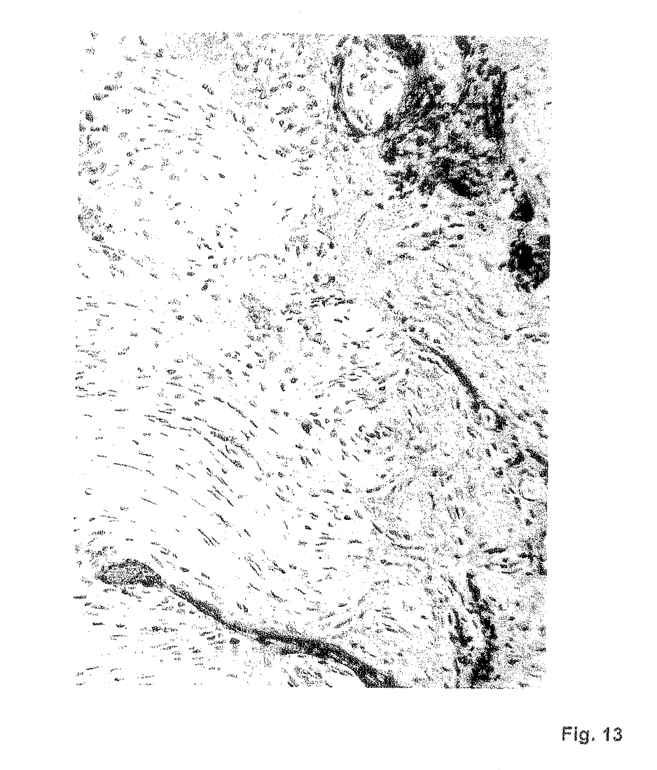

FIG. 14 intensity of the immunohistochemical staining for FZD 9 in a breast carcinoma sample.

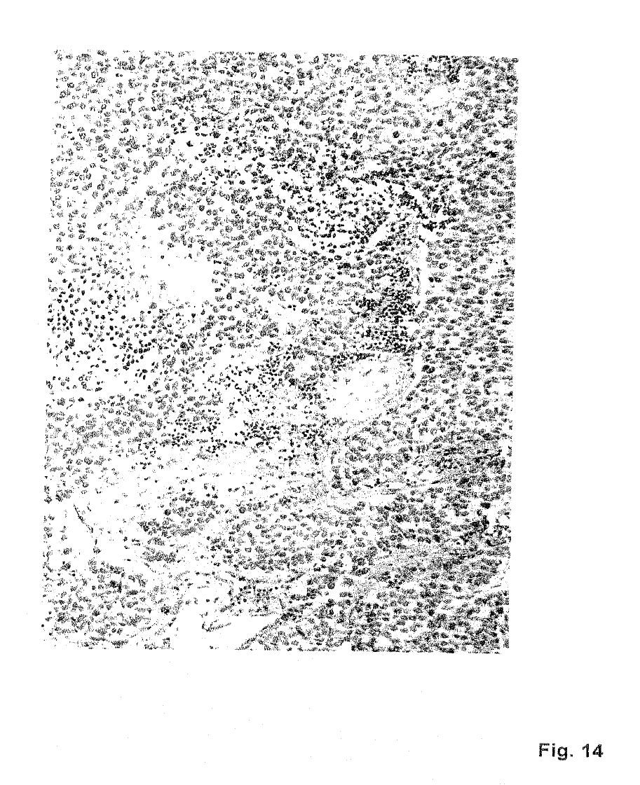

FIG. 15 intensity of the immunohistochemical staining for FZD 9 in a hemangioblastoma sample.

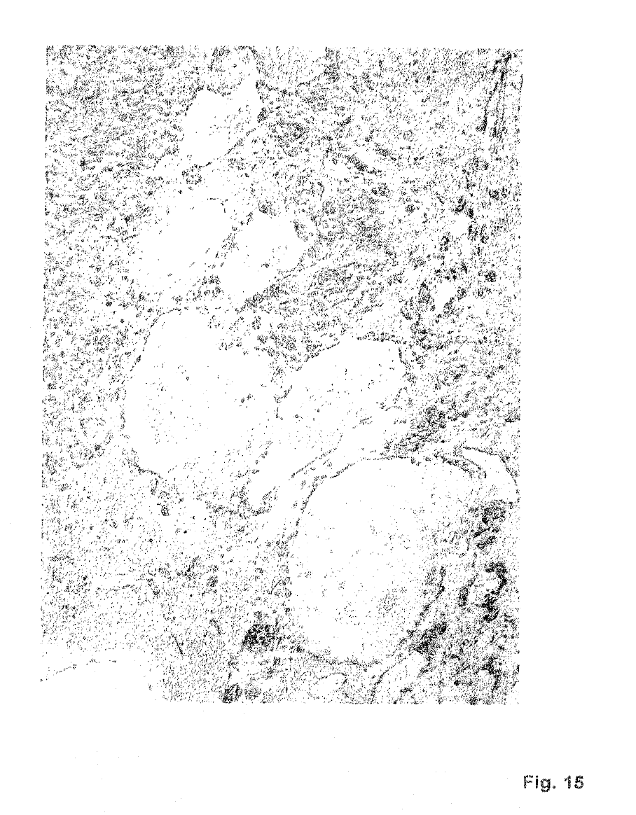

### DESCRIPTION OF PREFERRED EMBODIMENTS

### Exemplary Embodiment

### 1. The Wnt/FZD Signaling Pathway

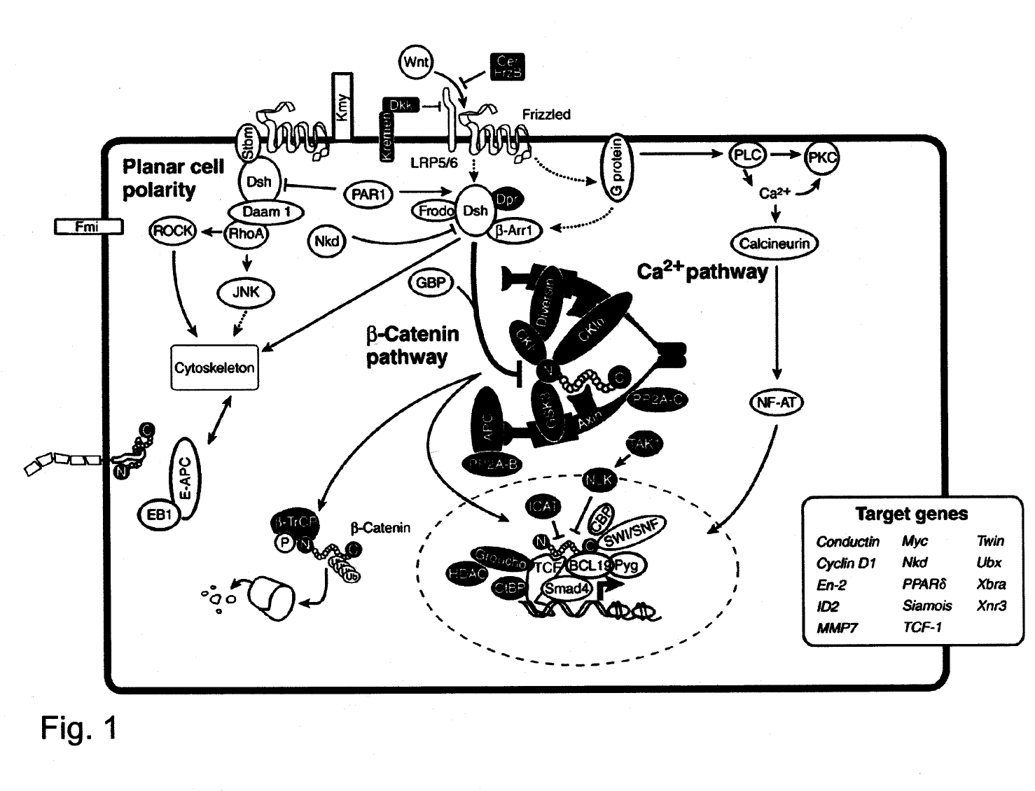

FIG. 1 shows schematically the Wnt/FZD signaling pathway in accordance with the publication by Huelsken J. and Behrens J., “The Wnt signaling pathway”, Journal of Cell Science 2002; 115:3977-3978. The contents of this publication are expressly incorporated in the present application documents by reference. The three branches of the Wnt/FZD signaling pathway are shown, namely the β-catenin signaling pathway (“β-catenin pathway”; middle branch), the Ca²⁺ signaling pathway (“Ca²⁺ pathway”; right-hand branch) and the planar cell polarity-regulating signaling pathway (“planar cell polarity”; left-hand branch). Besides the Frizzled receptor depicted at the top in the schematic cell membrane, it is possible to use indirectly the individual factors of the three signaling pathways, which are depicted schematically as circular or oval symbols, for example Dsh, Frodo, β-Arr1, PLC, PKC, JNK etc, and the Wnt protein, for identifying the substructures of the human brain.

### 2. MATERIAL AND METHODS

### 2.1 Patients

### (a) Astrocytomas

25 adults were investigated, including ten controls with normal brain, 7 WHO grade II astrocytomas, 9 WHO grade III astrocytomas, and 9 WHO grade IV astrocytomas. Details of these pathologies are indicated in Tables 1 and 2. The histological diagnosis and classification were carried out by two experienced neuropathologists in accordance with the WHO classification system. The tissue was obtained in compliance with the ethical guidelines of Tübingen University.

### (b) Further Brain Tumors

29 samples were acquired from the Institute of Brain Research (Tübingen) and the Pathology Institute (Katharinen-hospital Stuttgart) for investigating further brain tumors. The samples comprised 23 classical medulloblastomas, three desmoplastic medulloblastomas, two supratentorial PNETs and one medullo-myoblastoma (17 male and 12 female patients ranging in age from 2 to 64 years, average age 15.3 years). The histological diagnosis and classification took place in accordance with the WHO classification system. Further samples comprised meningiomas, schwannomas, hemangioblastomas and neurofibromatomas. Inspection of the clinical data showed recurrence of the tumor after a surgical procedure in six patients (Table 3). No cases of Turcot or Gorlin's syndromes were included. Samples from ten normal brains served as control. The regions of interest included the frontal lobe, the motor cortex, the hippocampus, the occipital lobe and cerebellum. Frozen tissue from the frontal lobe and the cerebellar hemispheres was prepared for the PCR. The tissue was obtained in compliance with the ethical guidelines of Tübingen University.

### (c) Non-Brain Tumors

Samples from institutes of Tübingen University hospital were obtained in compliance with the ethical guidelines of Tübingen University for investigating prostate carcinomas and breast carcinomas.

### 2.2 α-FZD 9 Antibodies

### 2.2.1 Monoclonal A-FZD 9 Antibody

The specific monoclonal antibody which is directed against human FZD 9 and which is produced by the W3C4 E11 hybridoma cells was obtained by immunizing four to eight-week old male Balb/c mice with an FZD 9-expressing retinoblastoma cell line WERI-RB-1 (acquired from the Deutsche Sammlung für Mikroorganismen und Zellkulturen, DSMZ, Braunschweig, Germany) which was cultured in RPMI1640 medium to which 10% fetal calf serum was added. The mice received intraperitoneal injections of 10⁷ cells five times at 2-week intervals. Four days after the last boost, the spleen was removed for fusion with the SP2-0 myeloma cell line. The resulting hybridomas were maintained in RPM11640 medium (GIBCO) comprising 10% fetal calf serum and hypoxanthine-aminopterin thymidine (HT; Sigma-Aldrich, Munich, Germany). Culture supernatants which were positive for WERI-RB-1 cells were screened for peripheral blood (B) and bone marrow (BM) cells. Hybridoma cells which secreted antibodies which were nonreactive for these cells were selected, cloned twice by limiting dilution and cultured in the presence of hypoxanthine-thymidine (HT; Sigma). The clone W3C4E11 complied with these criteria and was selected. The InM isotype of the resulting monoclonal antibody of the W3C4E11 cell line was determined by ELISA (Boehringer Mannheim, Mannheim, Germany). The specificity of W3C4E11 antibodies for human FZD 9 was confirmed by the selective recognition of human embryonic kidney cells (HEK-293) which had been transfected with the complete human FZD 9 gene. W3C4E11 recognizes untransfected HEK-293 cells or HEK-293 cells which have been transformed with FZD 1, 2, 4, 5, 7 or 10.

The W3C4E11 cell line was deposited on Jul. 15, 2004, at the Deutsche Sammlung für Mikroorganismen und ZellKulturen (DSMZ), Mascheroder Weg 1b, 38124 Braunschweig, under the number ACC2668.

### 2.2.2 Polyclonal α-FZD 9 Antibody

A polyclonal rabbit antibody against human FZD 9 (Acris Antibodies GmbH, Hiddenhausen, Germany, dilution 1:500) was used to investigate further brain tumors (non-astrocytomas) and non-brain tumors. The commercially available antibody clone SP4153P is directed against the first extracellular loop of human FZD 9.

### 2.3 Immunohistochemistry

### (a) Astrocytomas

Brain samples were removed surgically, immediately fixed in buffered paraformaldehyde and embedded in paraffin. For immunostaining, representative serial 3 μm sections were prepared. After the sections had been deparaffinized they were boiled in citrate buffer (2.1 g of sodium citrate per liter, pH 6) in a 600 watt microwave oven for 15 min. The endogenous peroxidase was inhibited with 1% H₂O₂ in methanol for 15 min. The sections were incubated in 10% normal pig serum (Biochrome, Berlin, Germany) in order to block the nonspecific binding of the immunoglobulins and were then incubated with the primary antibody. The monoclonal antibody derived from W3C4E11 described above was used for FZD 9 staining. In addition, a further polyclonal rabbit antibody against human FZD 9 was used (Acris Antibodies GmbH, loc. cit.; diluted 1:500). Microvascular endothelial cells were stained with anti-CD34 monoclonal antibody (diluted 1:50, Biomedicals, Augst, Switzerland), and Ki67 was stained with MIB-1 in order to show the cell proliferation (1:100, DAKO, Hamburg, Germany).

The antibody binding to the tissue sections was visualized using a biotinylated pig anti-rabbit (DAKO, Hamburg, Germany) or rabbit anti-mouse IgG F(ab)₂ antibody fragment. The sections were then incubated with a streptavidinavidin-biotin complex (DAKO, Hamburg, Germany), followed by development with 3,3′-diaminobenzidine (DAB) as chromogen (Fluka, Neu-Ulm, Germany). Finally, the sections were counterstained with hematoxylin.

After the immunostaining, the level of expression of FZD 9 was quantified by two methods. The total immunoreactivity of FZD 9 of each sample was graded by using a semiquantitative assessment introduced by “Sinicrope F A et al., “Bcl-2 and p53 oncoprotein expression during colorectal tumorigenesis”. Cancer Res. 1995; 55:237-241, Briefly stated, four categories were defined as follows: 0, completely negative; 1, weakly positive; 2, moderately positive; and 3, strongly positive. The density of the FZD 9 microvessels was examined under the light microscope using a method which was introduced by Weidner N. et al., “Tumor angiogenesis and metastasis-correlation in invasive breast carcinoma”. N. Engl. J. Med. 1991; 324:1-8. Briefly stated, the complete section was scanned with 40× magnification for the regions of highest FZD 9⁺ vascular density (hot spot), followed by counting the number of FZD 9⁺ vessels within a single field at 200× magnification (high power field, HPF) in each hot spot. Three hot spots were counted, and the average was calculated. The final results are expressed as arithmetic mean of the FZD 9⁺ positive vessels per HPF with standard error of the means (SEM). The intensity of FZD 9 expression and the density of the FZD 9⁺ microvessels were carefully ascertained by independent people.

Microvessel density (MVD) counts were determined by ascertaining the average number of hollow-lumen vessels stained by CD34 in 3 HPF using the same method employed for counting the density of FZD 9⁺ microvessels. The density of Ki67⁺ microvessels was determined by the same method used for counting the density of FZD 9⁺ microvessels.

### (b) Further Brain Tumors

Optimization of the polyclonal rabbit antibody was carried out both on cryostat sections and on paraffin sections, with identical results, for immunohistochemical detection of FZD 9. Consequently, all further studies were carried out on tissue samples embedded in paraffin. After the sections had been deparaffinized they were boiled in citrate buffer (see above) in a 600 watt microwave oven for 15 minutes. the endogenous peroxidase was inhibited with 1% H₂O₂ in methanol for 15 minutes. The sections were incubated in 10% normal pig serum (see above) in order to block the nonspecific binding of the immunoglobulins. The polyclonal rabbit antibody against human FZD 9 (Acris Antibodies GmbH, loc. cit., dilution 1:500) was used for FZD 9 staining. The antibody binding to the tissue sections was visualized with a biotinylated anti-rabbit pig antibody (DAKO, Hamburg, Germany), followed by incubation with a streptavidin-avidin-biotin complex (DACO, Hamburg, Germany). The chromogen used was 3,3′-diaminobenzidine (DAB) (Fluka, Neu-Ulm, Germany). Finally, the sections were counterstained with hematoxylin and embedded. The primary antibody was omitted from the controls.

The immunohistochemical tissue staining for MIB-1 was carried out on 4 μm-thick, formalin-fixed and paraffin-embedded samples using the benchmark immunohistochemistry system (Ventana, Tasken, Ariz., USA). The automated protocol is based on an indirect biotin-avidin system. Optimization of the MIB-1 antibody (1:100, DAKO, Hamburg, Germany) comprised pretreatment for cell conditioning for 6 minutes. After incubation of the primary antibody for 2 minutes, an avidin blocker and a biotin blocker was added for 4 minutes, followed by a nonspecifically biotinylated immunoglobulin secondary antibody and a diaminobenzidine substrate for visualization. The sections were then washed, counterstained with hematoxilin and embedded. The negative control sections were processed in parallel with each staining batch. All the histological investigations and photographic documentations took place using an Olympus AX70 microscope; the photographs were digitized and optimized for brightness and contrast in the print.

### (c) Non-Brain Tumors

Immunohistochemical detection of non-brain tumors took place starting from paraffin sections as described in (b).

### 2.4 Cell Culture and Treatment

### (a) Astrocytomas

Human cerebromicrovascular endothelial cells (HCEC), which were kindly provided by Prof. A. Muruganandam, are described in the prior art; cf. Muruganandam A. et al., “Development of immortalized human cerebromicrovascular endothelial cell line as an in vitro model of the human blood-brain barrier”. FASEB J. 1997; 11:1187-1197. These HCEC cells were cultured in RPMI-1640 medium with 10% heat-inactivated fetal calf serum (FCS) with penicillin and streptomycin at 100 U/ml (Gibco Grand Island, N.Y.) at 37° C. in 5% CO₂. In the cobalt chloride experiments, HCEC cells which had reached approximately 80% confluence were exposed to CoCl₂ (100 μmol) for the stated periods; cf. Cho, J., et al., “Cobalt chloride-induced estrogen receptor alpha down-regulation involves hypoxia-inducible factor-1alpha in MCF-7 human breast cancer cells”. Mol. Endocrinol. 2005; 19:1191-1199, The medium was renewed 24 hours before exposure to CoCl₂. Parallel samples in triplicate were prepared for each time point.

### (b) Further Brain Tumors

A representative human medulloblastoma cell line, DAOY, which shows expression of neuronal and glial elements, cf. Karasawa et al. (2002), J. Biol. Chem. 277; pages 37479-37486, was kindly provided by Dr Mittelbronn. These cells were cultured in minimal essential medium (Eagle) with 10% heat-inactivated fetal serum (FCS) with penicillin and streptomycin at 100 U/ml (Gibco, Grand Island, N.Y.) at 37° C. in 5% CO₂. Harvesting took place at approximately 80% confluence.

### 2.5 Real-Time Reverse Transcription PCR Analysis

### (a) Astrocytomas

Total RNA from cultured cells was prepared using the RNeasy mini kit (QIAGEN GmbH, Hilden, Germany) as stated by the manufacturer. 2 μg of RNA was reverse-transcribed into cDNA using randomized primers. FZD 9 mRNA expression by HCEC cells was then quantified using a real-time PCR in which SYBR green was used as detection reagent and 18 S ribosomal RNA was used as intermediate reference standard. The following primers were used: human 18S ribosomal RNA (sense, CGGCTACCACATCCAAGGAA; antisense, GCTGGAATTACCGCGGCT) and human FZD 9 (sense, GCAGTAGTTTCCTCCTGACCG; antisense TCTCTGTGTTGGTGCCGCC). The PCR was carried out in a real-time icycler (Biorad., Munich, Germany). All the reactions were carried out in a total volume of 15 μl which included 2× SYBR® green PCR mastermix and optimized primer concentrations. The cycling was started at 95° C. for 15 minutes, and this was then followed by 35 cycles of: 94° C. for 45 sec., 52° C. for 30 sec, and 72° C. for 45 sec. with a fluorescence detection at 72° C. The melting curve analysis was carried out between 50 and 100° C. with 0.5° C. intervals. Each sample was measured in triplicate, and the arithmetic mean was calculated.

### (b) Further Brain Tumors

Total RNA was prepared from cultured cells and frozen tissue samples using the peqGold TriFast kit (Peqlab Biotechnologie GmbH, Erlangen, Germany) was of the manufacturer's statements. For each sample, 2 μg of RNA under-went reverse transcription into cDNA using randomized primers. Real-time PCR was used to quantify the FZD 9 mRNA expression in DAOY cells. SYBR® green was used for detection. Human ribosomal 18S RNA served as internal reference standard (forward: CGGCTACCACATCCAAGGAA; reverse: GCTGGAATTACCGCGGCT). The following primer pair was used for FZD 9: forward: GCAGTAGTTTCCTCCTGACCG; reverse: TCTCTGTGTTGGTGCCGCC. All the reactions were used in a real-time icycler (Biorad., Munich, Germany) using a SYBR® green PCR mastermix and an optimized cycle protocol: 95° C. for 15 minutes, then 35 cycles of: 94° C. for 45 seconds, 52° C. for 30 seconds and 72° C. for 45 seconds with a fluorescence detection at 72° C. The melting curve analysis took place between 50 and 100° C. with 0.5° C. intervals.

### 2.6 Evaluation and Statistics

### (a) Astrocytomas

The number of FZD 9⁺ microvessels/HPF, MVD and Ki67⁺ microvessels/HPF are indicated as arithmetic means with standard errors of the means (SEM). Statistical analysis of the quantitative FZD 9 expression was carried out by one-way ANOVA followed by Dunnett's Multiple Comparison Tests (Grap Pad Prism 4.0 Software). The correction analysis was evaluated by checking Pearson's correlation coefficient. Significant levels were set at p<0.05 for all the statistical analyses.

### (b) Further Brain Tumors

The overall immunoreactivity for FZD 9 of each sample was graded using a semiquantitative evaluation which was introduced by Sinicrop; cf. Sinicrop et al. (1995), Cancer Res. 55, pages 237-241. The four categories were defined as follows: 0 completely negative; 1 weakly positive; 2 moderately positive; and 3 strongly positive. The MIB-1/Ki67 labeling index as the percentage proportion of cells in a tissue stain for Ki67 was evaluated by averaging the percentage proportion of Ki67⁺ nuclei counted in three high power fields. For this averaging, spots were selected after scanning the entire sample, and were counted and the average was determined. All the investigations were carried out independently by two people. The FZD 9 staining value and the Ki67 labeling index are indicated as arithmetic means with standard deviations (SEM). Statistical analysis of the quantitative FZD 9 expression took place by one-way ANOVA, followed by Dunnett's Multiple Comparison Test (Grap Pad Prism 4.0 Software). The correction analysis was evaluated by checking Pearson's correlation coefficient. The significance levels were set at p<0.05 for all the statistical analyses.

### 3. RESULTS

3.1 Preparation of the Specific Monoclonal Antibody Directed Against Human FZD 9 and Derived from W3C4E11 Cells

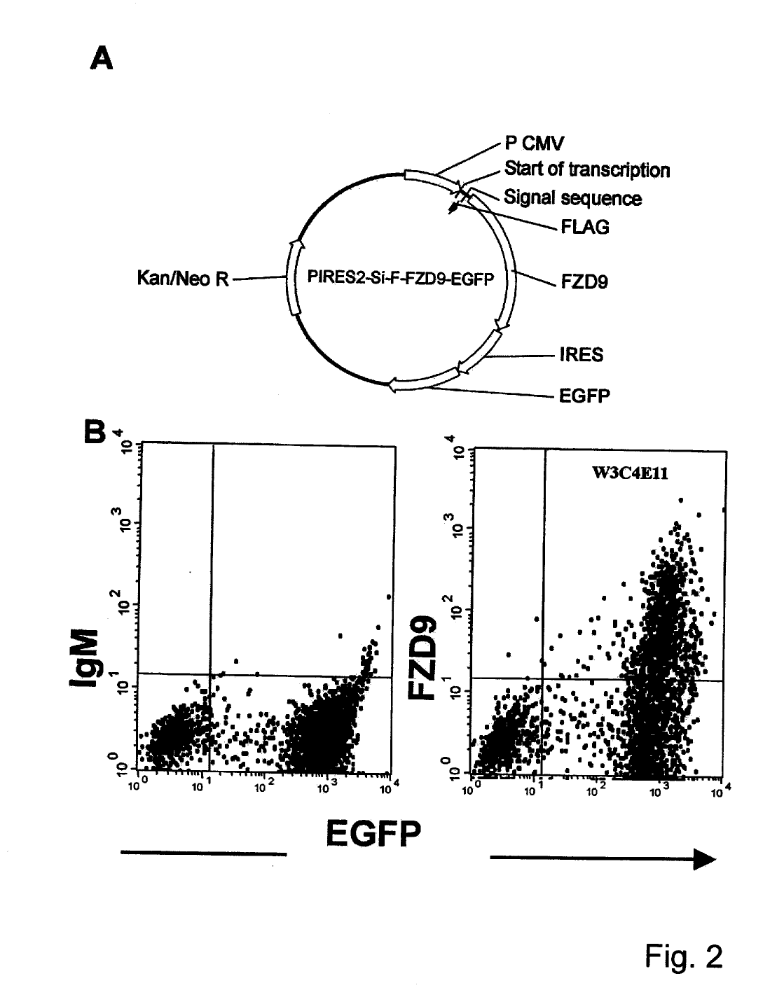

In order to analyze human FZD 9 protein expression, a monoclonal antibody against membrane-bound FZD 9 was obtained by immunizing a mouse with the FZD 9-expressing cell line WERI-RB-1. A transfected cell was produced in order to select potential FZD 9-reactive antibodies. For this purpose HEK-293 cells were transfected with a pIRES plasmid which comprised the human FZD 9 gene and a Flag tag at the N terminus (FIG. 2A). After three rounds of selecting cells which could be stained with an anti-Flag antibody using a cell sorter, most of the cells stably expressed the Flag tag on the cell surface. FIG. 2B shows that the W3C4E11 antibody reacts with the transfected cell, whereas wild-type HEK-293 cells were unreactive (not shown).

### 3.2 Expression of FZD 9 in Neuropathologically Normal Adult Human Brains

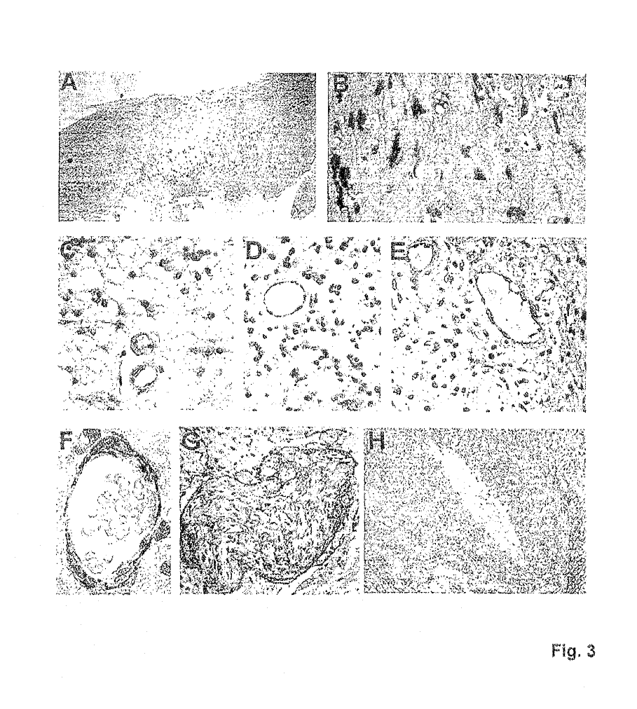

3.2.1 Although expression of FZD 9 in brain has been described, its expression pattern in the adult human brain is unknown as yet. Immunohistochemical investigations were therefore carried out on ten sections of paraffin-embedded normal human adult brains in order to determine the level of expression and the localization of FZD 9. FZD 9 expression was low in most sections (Table 1), and a few FZD 9⁺ neurons or glia were observed only occasionally. However, in sections containing hippocampal tissue, strong FZD 9 expression was observed in the hippocampal neurons (FIGS. 3A and B).

In some cases, FZD 9 was expressed on the endothelial cells of the large vessels located in the leptomeninx outside the brain parenchyma. Few FZD 9⁺ microvessels were observed in normal brains (Table 1).

TABLE 1
Clinical and histological data of control patients with normal brain

| | | | | Ki67 | | |
|---|---|---|---|---|---|---|
| | | Neuropathological | FZD9 | labeling | FZD9⁺ | |
| | Sex/ | diagnosis/pathological | stain | index | microvessels/ | |
| Case | age | diagnosis | intensity^{a} | (%) | HPF^{b} | MVD^{c} |
| 1 | F^{d}/82 | Normal brain/ | 1 | <0.01 | 4 ± 0.6 | 5 ± 1.5 |
| | | intrabdominal | | | | |
| | | bleeding, bypass | | | | |
| 2 | M^{e}/73 | Normal brain/ | 1 | <0.01 | 0.7 ± 0.3 | 1 ± 0 |
| | | pulmonary embolism | | | | |
| 3 | M/21 | Normal brain/ | 1 | <0.01 | 1 ± 0 | 1 ± 0 |
| | | fractured collarbone | | | | |
| 4 | F/49 | Normal brain with | 0 | <0.01 | 0.7 ± 0.3 | 1 ± 0 |
| | | signs of arteriosclerosis/ | | | | |
| | | cyst kidney, cyst | | | | |
| | | liver | | | | |
| 5 | F/92 | Normal brain/ | 0 | <0.01 | 1 ± 0 | 1.3 ± 0.3 |
| | | fulminant colitis | | | | |
| 6 | F/75 | Incipient cerebral | 1 | <0.01 | 1.7 ± 0.3 | 8.3 ± 3 |
| | | edema/cirrhosis of | | | | |
| | | the liver | | | | |
| 7 | F/39 | Normal brain/ | 0 | <0.01 | 1 ± 0 | 13 ± 1 |
| | | pulmonary embolism | | | | |
| 8 | M/25 | Normal brain/ | 0 | <0.01 | 1 ± 0 | 1.3 ± 0.3 |
| | | rupture of aorta | | | | |
| 9 | M/51 | Normal brain/ | 0 | <0.01 | 1 ± 0 | 1.7 ± 0.3 |
| | | stenosis of femoral | | | | |
| | | arteries | | | | |
| 10 | F/36 | Normal brain with | 1 | <0.01 | 4.3 ± 0.3 | 19.3 ± 2.4 |
| | | signs of anemia/ | | | | |
| | | carcinoma of the | | | | |
| | | pleural cavity | | | | |

^{a}The stain intensity was divided into four categories: 0, completely negative; 1, weakly positive; 2, moderately positive; and 3, strongly positive;
^{b}HPF = high power field;
^{c}MVD = microvessel density;
^{d}F = female;
^{e}M = male.

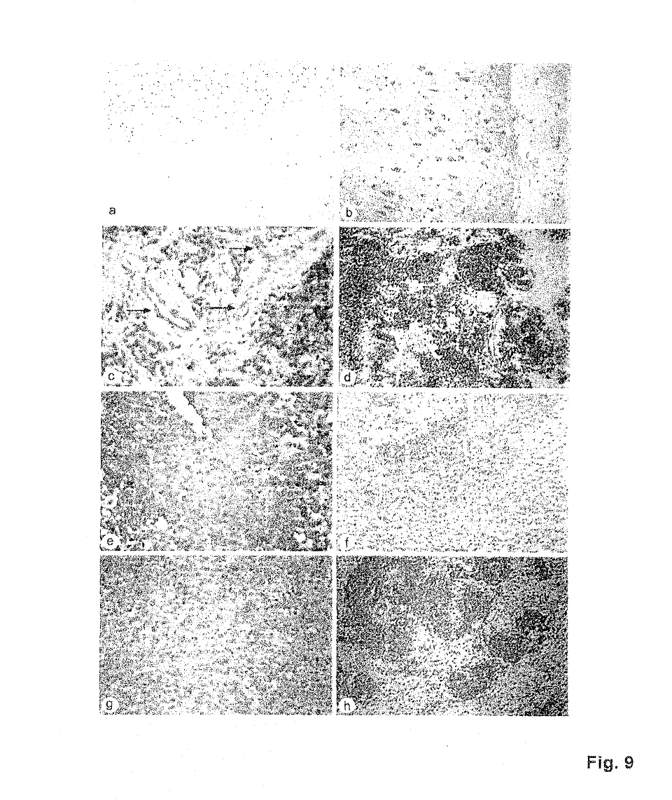

3.2.2 In a further approach, samples from various regions of 10 normal adult human brains were stained likewise in order to determine the level of expression of FZD 9 and the localization of FZD 9, FZD 9 expression in most regions was low; a few FZD 9⁺ neurons or glia were observed occasionally. FZD 9 expression in the cerebellum was confined to the endothelial cells of the microvessels (FIG. 9A).

In contrast thereto, strong FZD 9 expression was observed in the pyramidal layers of the hippocampal neurons (FIG. 9B). In the leptomeningides, FZD 9 was expressed on the endothelial cells of the large vessels. All the negative controls showed no staining.

3.3 Expression of FZD 9 in Human Tumors

3.3.1 Astrocytomas

In parallel with the investigation of normal or healthy brains, FZD 9 expression was investigated in human astrocytomas with WHO grade II to IV. FZD 9 expression was observed both in microvascular endothelial cells and in neo-plastic cells (FIG. 3C-E).

Expression of FZD 9 in microvascular endothelial cells was observed in human astrocytomas (FIG. 3C-F). The density of FZD 9⁺ microvessels was high in malignant astrocytomas (40.1±21.3 for WHO grade III and 91.37±17.79 for WHO grade IV) and low in lower high-grade astrocytomas (2.1±1.0 for WHO grade II) (Table 2).

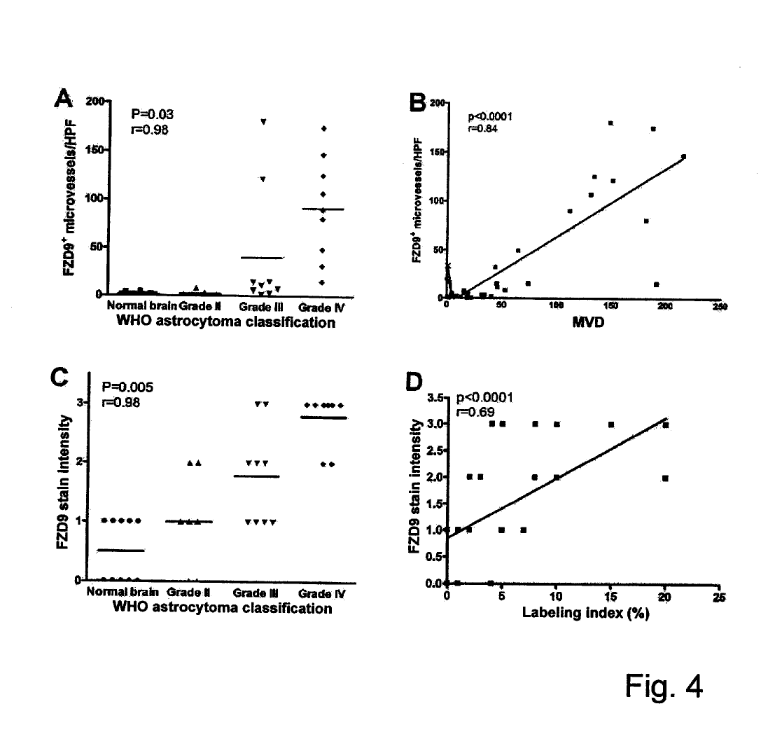

The density of FZD 9⁺ microvessels in glioblastomas was significantly higher than in WHO grade II astrocytomas (p<0.001) and in normal brains (p<0.001) (FIG. 4A and Table 2). Multilayer microvascular proliferations (‘glomeroid tufts’) of proliferating capillaries are a characteristic feature of the microvascular structure of glioblastomas. These microvascular proliferations comprise vascular channels which are formed by layers of endothelial cells and are separated by incomplete layers of pericytes; cf. Kleinhues P. et al., “The WHO classification of tumors of the nervous system”. J. Neuropathol. Exp. Neurol. 2002; 61:215-225. The multilayer microvascular proliferations likewise showed strong expression of FZD 9 (FIG. 3G).

FZD 9 expression in tumor cells was observed in one of seven WHO grade II astrocytomas, in five of nine WHO grade III astrocytomas, and in nine of nine samples of WHO grade IV astrocytomas, from which it may be concluded that FZD 9 is expressed substantially more strongly in tumor cells of malignant astrocytomas than in those of lower-grade astrocytomas. The overall level of FZD 9 immunoreactivity was semiquantified. FZD 9 expression was high in WHO grade III astrocytomas (p<0.01, compared with normal brains) and WHO grade IV astrocytomas (p<0.001, compared with normal brains) and low in WHO grade II astrocytomas (p>0.05, compared with normal brains) (FIG. 4A and Table 2). FZD 9 expression in human glioblastomas was very heterogeneous. A further histopathological characteristic of glioblastomas is the presence of necroses with pseudopalisades (‘pseudopalisading necroses’), i.e. a central necrosis with perinecrotic astrocytoma cells, which frequently assume a pseudopalisade pattern; cf. Kleinhues P. et al., (loc. cit.). Strong FZD 9 expression was observed in the astrocytoma cells in necroses with pseudopalisades in the present investigations (FIG. 3H).

TABLE 2
Clinical and histopathological data for astrocytoma patients

| | | | | Ki67 | | |
|---|---|---|---|---|---|---|
| | | | FZD9 | labeling | FZD9⁺ | |
| | Sex/ | | stain | index | microvessels/ | |
| Case | age | Diagnosis | intensity^{a} | (%) | HPF^{b} | MVD^{c} |
| 11 | F^{d}/49 | Ast^{f} Grade II | 1 | 1 | 1 ± 0 | 33.3 ± 3.3 |
| 12 | M^{e}/65 | Ast Grade II | 0 | 1 | 1 ± 0 | 4 ± 0.6 |
| 13 | F/25 | Ast Grade II | 0 | 4 | 0 | 18.7 ± 0.9 |
| 14 | M/43 | Ast Grade II | 2 | 3 | 8 ± 1.2 | 53.5 ± 24.5 |
| 15 | M/39 | Ast Grade II | 1 | 1 | 0.3 ± 0.3 | 22.3 ± 3.4 |
| 16 | F/36 | Ast Grade II | 2 | 2 | 3.3 ± 0.3 | 32 ± 7.1 |
| 17 | M/37 | Ast Grade II | 1 | 1 | 1.3 ± 0.3 | 6.5 ± 0.5 |
| 18 | M/39 | Ast Grade III | 3 | 4 | 121.7 ± 5.8 | 151 ± 11.5 |
| 19 | M/67 | Ast Grade III | 1 | 5 | 5.7 ± 1.8 | 16 ± 1.5 |
| 20 | F/49 | Ast Grade III | 2 | 20 | 14.67 ± 2.3 | 46 ± 8.6 |
| 21 | F/23 | Ast Grade III | 2 | 20 | 15 ± 1.7 | 74.7 ± 9.3 |
| 22 | M/48 | Ast Grade III | 2 | 10 | 7.5 ± 0.5 | 15.7 ± 1.2 |
| 23 | F/44 | Ast Grade III | 1 | 2 | 1.3 ± 0.3 | 40.7 ± 4.9 |
| 24 | F/69 | Ast Grade III | 1 | 7 | 3 ± 0.6 | 34.7 ± 2.6 |
| 25 | F/40 | Ast Grade III | 1 | 5 | 11 ± 0.6 | 46.3 ± 8.1 |
| 26 | M/58 | Ast Grade III | 3 | 15 | 181 ± 8.1 | 315 ± 13.2 |
| 27 | M/50 | GB^{g} Grade IV | 3 | 20 | 125.7 ± 8.1 | 133.7 ± 5.4 |
| 28 | M/58 | GB Grade IV | 3 | 8 | 80.67 ± 1.4 | 181.7 ± 38.3 |
| 29 | F/68 | GB Grade IV | 2 | 8 | 32 ± 2.6 | 44.3 ± 2.8 |
| 30 | F/93 | GB Grade IV | 2 | 10 | 15 ± 3 | 192 ± 9.2 |
| 31 | F/61 | GB Grade IV | 3 | 20 | 49.3 ± 1.4 | 65 ± 2.9 |
| 32 | M/68 | GB Grade IV | 3 | 5 | 107 ± 4 | 131 ± 6.4 |
| 33 | F/49 | GB Grade IV | 3 | 10 | 147 ± 3.2 | 215.3 ± 3.4 |
| 34 | F/51 | GB Grade IV | 3 | 15 | 90.3 ± 5.8 | 112 ± 19.6 |
| 35 | M/52 | GB Grade IV | 3 | 8 | 175 ± 5.1 | 187.3 ± 5.6 |

^{a}The stain intensity was divided into four categories: 0, completely negative; 1, weakly positive; 2, moderately positive; and 3, strongly positive;
^{b}HPF = high power field;
^{c}MVD = microvessel density;
^{d}F = female;
^{e}M = male;
^{f}Ast = astrocytoma;
^{g}GB = glioblastoma.

3.3.2 Correlation Between the Immunoreactivity of FZD 9 with the Astrocytoma Grade, the Microvessel Density and the Cell Proliferation

Since the density of FZD 9⁺ microvessels was high in malignant astrocytomas and low in lower-grade astrocytomas, the correlation of the density of FZD 9⁺ microvessels with the WHO astrocytoma classification and the astrocytoma microvessel density (MVD) was analyzed as a measure of the degree of angiogenesis. As shown in FIG. 3A, there was a positive correlation between the density of FZD 9⁺ microvessels with the WHO astrocytoma classification (p=0.03, r=0.98). The correlations between the density of FZD 9⁺ microvessels and the MVD in astrocytomas was evaluated using Pearson's correlation coefficient. The MVD was measured with a generally used method using CD34 immunostaining. A close correlation between the density of FZD 9⁺ microvessels and the MVD was observed in human astrocytomas (FIG. 4B; p<0.0001, r=0.84).

In addition, the overall FZD 9 immunoreactivity was analyzed with the WHO astrocytoma classification and the astrocytoma proliferation activity. The FZD 9 stain intensity showed a positive correlation with the WHO astrocytoma classification with statistical difference (FIG. 4C, p=0.005, r=0.98). One of the main characteristics of neoplasms is a high level of cell proliferation. Ki67 is the most commonly used antigen for evaluating cell proliferation by means of immunostaining. Ki67 is a nuclear protein which is present in all non-GO phases of the cell cycle. In the present investigations, the Ki67 labeling index, the percentage proportion of cells in a tissue staining for Ki67, was used in order to investigate the astrocytoma proliferation. Significant correlations between the Ki67 labeling index and the intensity of the FZD 9 immunostaining were found in human astrocytomas (FIG. 4D; p<0.0001, r=0.69).

3.3.3 Expression of FZD 9 in Medulloblastomas, Primitive Neuroectodermal Tumors (PNETs) and Medullomyoblastomas

FZD 9 expression in the endothelium of tumor microvessels and large vessels was observed in 21 (72.4%) of the 29 investigated brain tumors (FIG. 9C, the vessels are labeled with arrows). Evaluation of the cytoplasmic expression using a semiquantitative evaluation revealed 5 strongly (value 3), 15 moderately (value 2) and 10 weakly positive tumors (value 1, traces 9D to G).

A diffuse expression pattern over the whole tissue sample without hot spots was observed in 26 (89.6%) brain tumors. An unequal distribution pattern was observed in four tumors, showing stronger FZD 9 expression in some regions with high cell density (FIG. 9H). No tumor sample remained unlabeled (value 0).

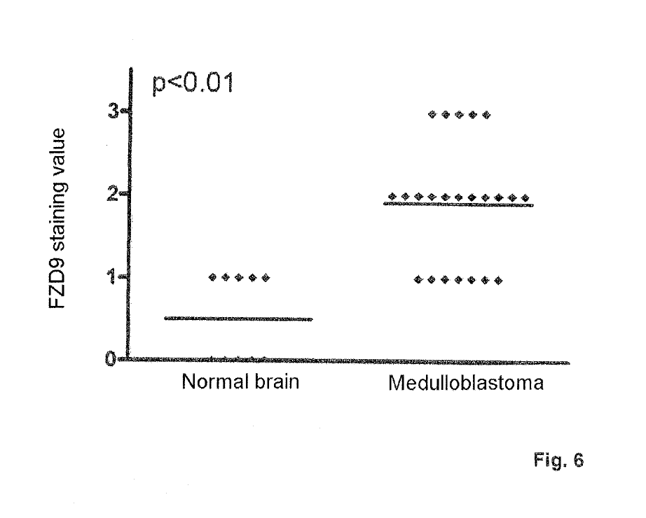

Consistent with its function as transmembrane protein, FZD 9 expression was also found on the cell membrane, which was frequently indistinguishable from the small cytoplasm. Compared with normal brain, the FZD 9 stain intensity, i.e. the FZD 9 expression pattern, was significantly higher (FIG. 6, p<0.01).

The one-way analysis of variants of the means between the classical desmoplastic and supratentorial variants showed no significant differences in expression. The evaluated expression levels were likewise independent of tumor recurrence, age or sex of the patient (p<0.05, data not shown).

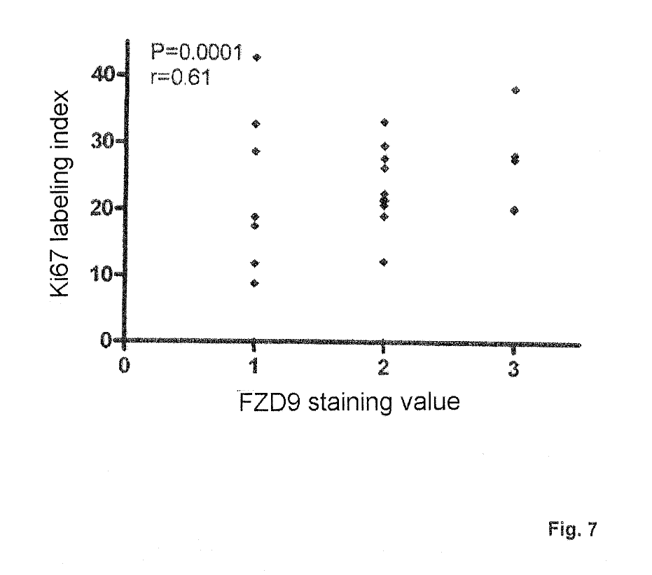

The MIB-1/Ki67 index is a conventional quantity for evaluating cell proliferation in tumors. As a nuclear protein, MIB-1/Ki67 is present in all non-GO phases of the cell cycle. An average Ki67 index of 93.6 was observed, the index being between 42.7 and 8.8 in the 29 investigated patients (Table 3). The normal brain controls showed only a few proliferating cells. Comparative analysis of the Ki67 and FZD 9 results revealed a significant correlation between FZD 9 immunostaining level and the Ki67 labeling index in medulloblastoma (FIG. 7, p<0.0001, R 0.61).

TABLE 3
Results for the level of the FZD 9 stain intensity and the MIB-1 (Ki67)
labeling index. The tumors labeled with an asterisk were selected
for real-time PCR.

| | | | | | | FZD 9 |
|---|---|---|---|---|---|---|
| | | | | | | stain |
| Case | Diagnosis | Sex | Age | Recurrence | MIB-1 (%) | intensity^{c} |
| 1* | classical | F | 2 | | 28.6 | 1 |
| 2 | classical | F | 4 | | 20.4 | 3 |
| 3 | classical | M | 5 | | 18.8 | 1 |
| 4 | classical | F | 5 | | 19.1 | 2 |
| 5 | classical | M | 6 | | 21.6 | 2 |
| 6 | classical | M | 7 | | 21.4 | 2 |
| 7 | classical | M | 8 | | 22.4 | 2 |
| 8 | classical | M | 8 | | 33.2 | 2 |
| 9 | classical | F | 9 | | 9.7 | 2 |
| 10 | classical | F | 90 | | 8.8 | 1 |
| 11 | classical | M | 11 | | 12.2 | 2 |
| 12 | classical | M | 12 | yes | 32.7 | 1 |
| 13 | classical | M | 13 | | 26.3 | 2 |
| 14 | classical | M | 15 | | 27.7 | 2 |
| 15 | classical | M | 15 | | 20.7 | 2 |
| 16 | classical | F | 20 | | 17.5 | 1 |
| 17 | classical | F | 20 | | 27.7 | 3 |
| 18 | classical | F | 20 | | 20.2 | 2 |
| 19 | classical | F | 20 | yes | 38.3 | 3 |
| 20 | classical | M | 33 | yes | 29.6 | 2 |
| 21 | classical | M | 37 | yes | 28.3 | 3 |
| 22 | classical | M | 64 | | 42.7 | 1 |
| 23 | classical | F | 39 | yes | 11.8 | 1 |
| 24 | desmoplastic | M | 20 | | 15.1 | 2 |
| 25 | desmoplastic | F | 20 | | 22.4 | 1 |
| 26 | desmoplastic | M | 33 | yes | 31.0 | 1 |
| 27 | medullomyoblastoma | M | 3 | | 40.3 | 2 |
| 28 | PNET | M | 4 | | 31.0 | 1 |
| 29 | PNET | F | 6 | | 9.9 | 3 |

F = female,
M = male.

Real-Time PCR

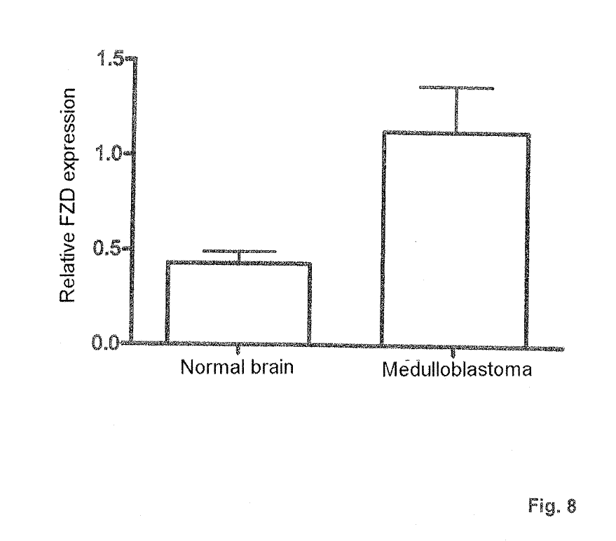

In order to confirm the immunohistochemical results, the FZD 9 mRNA expression was analyzed using real-time PCR in selected native medulloblastoma tissue samples (Table 3). These results were compared with samples from normal human brain. As in FIG. 8, the average FZD 9 expression in classical medulloblastomas was 2.1 times higher than in normal brains. In addition, FZD 9 mRNA expression was analyzed in a well-known medulloblastoma cell line, in DAOY cells. The relative FZD 9 mRNA expression in the DAOY cells cultured under normal conditions was increased up to seven-fold by comparison with the internal reference standard.

3.5 Expression of FZD 9 in Schwannomas, Hemangioblastomas, Meningeomas, Neurofibromatomas, Prostate Carcinomas and Breast Carcinomas

The immunohistochemical staining for FZD 9 which it was possible to find in the said tumors was throughout significantly greater than in control sections with healthy tissue. It is concluded therefrom that FZD 9 is distinctly overexpressed in these tumors too.

3.6 Up Regulation of FZD 9 Expression in Cobalt Chloride-Treated HCEC Cells

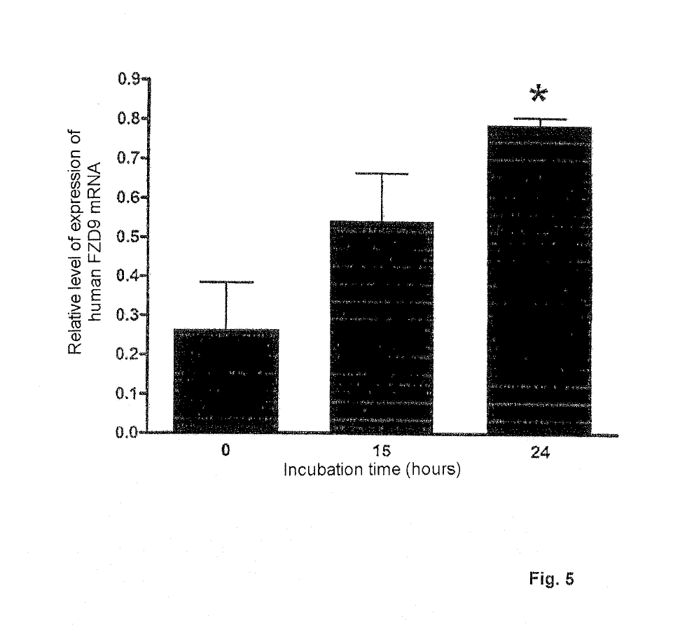

During the immunohistochemical investigations, FZD 9 expression was observed in microvascular endothelial cells, with a positive correlation between the density of the FZD 9 microvessels and the grade of the astrocytoma and the MVD. The inventors have thus realized that FZD 9 plays a role in astrocytoma angiogenesis. Hypoxia is a major inducer of angiogenesis. The inventors postulate that hypoxic stimulation stimulates FZD 9 expression in endothelial cells. In order to verify these effects of hypoxia on FZD 9 expression, HCEC brain endothelial cells were incubated with cobalt chloride which is known to be used as hypoxia imitator in cell cultures and which is known to activate the hypoxic signaling pathway through sterilization of the hypoxia-inducible transcription factor 1α; cf. Vengellur A., LaPres J. “The role of hypoxia inducible factor alpha in cobalt chloride induced cell death in mouse embryonic fibroblasts”. Toxicol. Sci, 2004; 82:638-646. After the incubation, FZD 9 expression was measured using real-time PCR with primers which were specific for FZD 9. FZD 9 expression was increased 15 hours after incubation with cobalt chloride (FIG. 5, two-fold higher than in untreated cells) and was greatly raised 24 hours after the incubation (FIG. 5; three-fold higher than in untreated cells, p<0.05). These results verify the findings obtained previously by the inventors with tissue sections, that FZD 9 is an important factor in (tumor) angiogenesis.

### 4. CONCLUSIONS

The inventors have surprisingly found that FZD 9 can be used to identify and, where appropriate, treat a wide variety of tumors. They therefore provide valuable diagnostic and therapeutic uses and methods.

Therefore, what is claimed, is:

1. Method for treating a tumor of a human or animal, the tumor being selected from the group consisting of: neurotumor/CNS tumor, prostate carcinoma and breast carcinoma, comprising the step of administering to the subject in need of such a treatment an amount sufficient to treat the tumor of a composition comprising an agent which binds to Frizzled 9 (FZD 9).

2. The method of claim 1, wherein the neurotumor/CNS tumor is selected from the group consisting of: glioblastomas including astrocytomas, medulloblastomas including primitive neuroectodermal tumors (PNET), schwannomas, hemangioblastomas, meningeomas and neurofibromatomas.

3. The method of claim 1, wherein the agent is selected from the group consisting of a polyclonal antibody, a monoclonal antibody, an antibody fragment which exhibits the binding properties of the antibody.

4. The method of claim 1, wherein the agent is a monoclonal antibody which is produced by the hybridoma cell W3C4E11 which was deposited on Jul. 15, 2004 in accordance with the Budapest treaty at the DSMZ under the number DSM ACC2668, or a fragment thereof.

5. The method of claim 3, wherein the antibody or the antibody fragment is a humanized antibody or a humanized antibody fragment.

6. The method of claim 4, wherein the antibody is a humanized antibody or a humanized antibody fragment.

7. The method of claim 3, wherein the antibody or the antibody fragment is coupled to a detectable marker or to a therapeutic substance.

8. The method of claim 3, wherein the antibody or the antibody fragment is coupled to an antitumor active substance.

9. A method for identifying tumors in a human or animal, comprising the following steps:
(1) providing a biological sample derived from the human or animal creature to be investigated;
(2) establishing the level of expression of FZD 9 in the biological sample;
(3) comparing the level of expression of FZD 9 established in step (2) with the level of expression in a biological sample from a healthy creature, and
(4) identifying a tumor if it is established in step (3) that the level of expression of FZD 9 in the biological sample of the creature to be investigated is significantly higher than the level of expression of FZD 9 in the biological sample of the healthy creature.

10. The method of claim 9, wherein the tumors are neurotumors/CNS tumors, and the biological samples include neural tissue.

11. The method of claim 9, wherein the tumors are selected from the group consisting of: glioblastomas including astrocytomas, medulloblastomas including primitive neuroectodermal tumors (PNET), schwannomas, hemangioblastomas, meningeomas and neurofibromatomas and prostate carcinomas and breast carcinomas.

12. The method of claim 9, wherein the biological samples are selected from prostate tissue and breast tissue.

13. The method of claim 9, wherein the level of expression is established at the level of the mRNA coding for FZD 9.

14. The method of claim 9, wherein the level of expression is established at the level of the FZD 9 protein.

15. The method of claim 9, wherein a polyclonal or monoclonal antibody which binds to FZD 9, or an antibody fragment thereof which exhibits the binding properties of the antibody, is used to establish the level of expression.

16. The method of claim 9, wherein the monoclonal antibody which is produced by the hybridoma cell W3C4E11 which was deposited on Jul. 15, 2004 in accordance with the Budapest treaty at the DSMZ under the number DSM ACC2668, is used as antibody.


SEQUENCE LISTING (4 sequences)

SEQ ID NO: 1 - DNA, 20 bp, Artificial sequence
  Synthetic oligonucleotide primer.
cggctaccacatccaaggaa

SEQ ID NO: 2 - DNA, 18 bp, Artificial sequence
  Synthetic oligonucleotide primer.
gctggaattaccgcggct

SEQ ID NO: 3 - DNA, 21 bp, Artificial sequence
  Synthetic oligonucleotide primer.
gcagtagtttcctcctgaccg

SEQ ID NO: 4 - DNA, 17 bp, Artificial sequence
  Synthetic oligonucleotide primer.
tctctgttggtgccgcc